(19) United States
(12) Patent Application Publication
(10) Pub. No.: US 2008/0210134 A1
(43) Pub. Date: Sep. 4, 2008

(54) Precipitated Calcium Carbonate (PCC) from Sugar Processing Byproducts for use in Cementitious Applications and Methods Thereof

(76) Inventors: Dallas A. Hanks, Burley, ID (US); Douglas Shaw, Santa Clara, UT (US)

Correspondence Address: GREENBERG TRAURIG LLP (LA), 2450 COLORADO AVENUE, SUITE 400E, INTELLECTUAL PROPERTY DEPARTMENT, SANTA MONICA, CA, 90404, US

(21) Appl. No.: 12/038,545

(22) Filed: Feb. 27, 2008

Related U.S. Application Data
(60) Provisional application No. 60/904,405, filed on Mar. 2, 2007.

(51) Int. Cl. C04B 22/10; B01D 35/28; C12M 1/00

(52) U.S. Cl. 106/464; 422/255; 435/289.1

(57) ABSTRACT

A process to make, in part, a refined material for use as crystallization accelerant in cementitious products. Evidence shows that calcium carbonate Fines (Ca⁺ ions) accelerate the crystallization process of CSH (Calcium-Silicate-Hydrate) phases which lends many advantages in cementitious products. The (PCC) fines materials from the sugar beet processing byproducts consist of these materials with various elements mixed or attached. This process selects for advantageous particles and discards others. This process includes, but is not limited to removing part of the undesirable material by either by screening or biodigestion of the lime to utilize energy contained in said unwanted material to dry in part said particles of PCC to specific moisture content and then screening the material to the remove more unwanted material and size PCC to spec necessary for cementitious materials.

### RELATED APPLICATIONS

This application claims the benefit of and full Paris Convention priority to U.S. Provisional Application Ser. No. 60/904,405, filed Feb. 28, 2007, entitled “Refinement of Precipitated Calcium Carbonate (PCC) from Sugar Processing Byproducts for use in Cementitious Applications,” the contents of which are incorporated by reference herein in its entirety.

### BACKGROUND

Sugar production from sugar beets requires the use of calcium carbonate (CaCO₃) for purifying sugar. To obtain calcium carbonate, raw limestone is heated until it becomes CaO and then added in combination with CO₂ to sugar the beet juices. The combination of the two products produces a chemical reaction resulting in calcium carbonate precipitate, referred to as precipitated calcium carbonate (PCC). The PCC byproduct is considered a waste product of the sugaring process and becomes a waste disposal issue on the order of 100,000's of tons per year. PCC is a colloid having wide variation in particle size and contains other contaminants, including sugar.

### SUMMARY

A process to make, in part, a refined material for use as crystallization accelerant in cementitious products. Evidence shows that calcium carbonate Fines (Ca⁺ ions) accelerate the crystallization process of CSH (Calcium-Silicate-Hydrate) phases which lends many advantages in cementitious products. The (PCC) fines materials from the sugar beet processing byproducts consist of these materials with various elements mixed or attached. This process selects for advantageous particles and discards others. This process includes, but is not limited to removing part of the undesirable material by either by screening or biodigestion of the lime to utilize energy contained in said unwanted material to dry in part said particles of PCC to specific moisture content and then screening the material to the remove more unwanted material and size PCC to spec necessary for cementitious materials.

According to a feature of the present disclosure, a method is disclosed comprising obtaining raw precipitated calcium carbonate (PCC), drying the PCC, screening the PCC to form a finished PCC, and providing the finished PCC to be used as a cementitious replacement. Extensions of the method including biodigesting the PCC.

According to a feature of the present disclosure, an apparatus is disclosed comprising a collector for collecting raw precipitate calcium carbonate (PCC) from a process producing as a waste product, a dryer for removing the moisture content of the PCC to a predetermined level, and a screener for obtaining a finished PCC having a predetermined particle size suitable as a cementitious replacement. The raw PCC is processed to be used as a cementitious replacement.

According to a feature of the present disclosure, a method is disclosed comprising obtaining raw precipitated calcium carbonate (PCC) from the sugaring process of sugar beets, drying the PCC to a moisture level of not greater than 10%, screening the PCC to form a finished PCC, providing the finished PCC as a cementitious replacement.

### DRAWINGS

The above-mentioned features and objects of the present disclosure will become more apparent with reference to the following description taken in conjunction with the accompanying drawings wherein like reference numerals denote like elements and in which:

FIG. 1 is a flow diagram of an embodiment of a method for creating a finished precipitated calcium carbonate (PCC) from raw PCC;

FIG. 2 is a block diagram of an embodiment of an apparatus and method of drying raw or partially finished PCC; and

FIG. 3 is a block diagram of an embodiment of a biodigester for the biodigestion of organic molecules in raw PCC.

### DETAILED DESCRIPTION

In the following detailed description of embodiments of the invention, reference is made to the accompanying drawings in which like references indicate similar elements, and in which is shown by way of illustration specific embodiments in which the invention may be practiced. These embodiments are described in sufficient detail to enable those skilled in the art to practice the invention, and it is to be understood that other embodiments may be utilized and that logical, mechanical, biological, electrical, functional, and other changes may be made without departing from the scope of the present invention. The following detailed description is, therefore, not to be taken in a limiting sense, and the scope of the present invention is defined only by the appended claims. As used in the present disclosure, the term “or” shall be understood to be defined as a logical disjunction and shall not indicate an exclusive disjunction unless expressly indicated as such or notated as “xor.”

The inventors have discovered that the larger colloids of PCC comprise the bulk of the contaminants; screening of these larger colloids reduces the contaminate level of the PCC enough to make the PCC usable as a partial cementitious material replacement.

Presently, mined and ground limestone is used as the basis for cementitious material in concrete and other products. The instant disclosure introduces an alternative to mined and ground limestone, which reduces destructive mining techniques, thereby being a more environmentally friendly process. Moreover, because PCC is a waste byproduct of the sugar purification process with sugar beets, the present disclosure eliminates much of the waste problem inherent in sugaring beets. The present disclosure expressly contemplates using PCC as a cementitious substitute in ready-mix, precast, shotcrete self-consolidating concrete, bricks, blocks, and pipe, although a person of ordinary skill in the art will appreciate that the present methods extend to a much greater range of products than those listed herein.

According to embodiments, a method is disclosed for refining PCC from sugar refining process so as to produce a material suitable for use as a cementitious material.

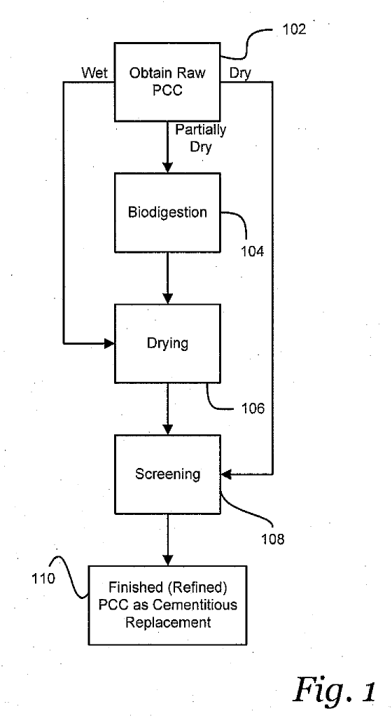

According to embodiments and as illustrated in FIG. 1, there is shown a method of processing raw PCC 102 which depends on the moisture content of raw PCC 102. Raw PCC 102 that is the byproduct of the sugaring process of sugar beets is either in solution (wet), dried, or partially dried. PCC is collected using a collector, which comprises any implement that can obtain raw PCC and transfer the raw PCC to apparatuses of the present disclosure for processing as a cementitious replacement. Such collectors, according to embodiments, include automated methods such as conveyor belt-like mechanisms, lead screws, pumps and conduits, manual collection and transport, and other ways of harvesting raw PCC and transporting the raw PCC to a location for use according to the present disclosure.

PCC may be dry when obtained in operation 102 prior to use in the methods of the present disclosure. Accordingly, dry PCC needs no drying and is directly screened, as described in greater detail below, in operation 108 to form a refined PCC to be used as a cementitious material concretes and cements, according to embodiments.

Likewise, the raw PCC may be partially dry when obtained in operation 102. Prior to the screening process of operation 108, the raw PCC must be dried enough to ensure the degree of refinement of the PCC is great enough to be suitably used as a cementitious replacement materials in cements and concretes. Therefore, the raw PCC is dried in operation 106. Drying may be accomplished in a number of ways. For example, drying may occur by exposing the raw PCC to air for a suitable length of time, whereby the moisture in the PCC is deposited in the air as a vapor and thus removed from the PCC. According to other embodiments, heat may be applied to the PCC to more rapidly vaporize the moisture in the PCC. Artisans will readily understand the various ways of drying a precipitate, including via chemical methods.

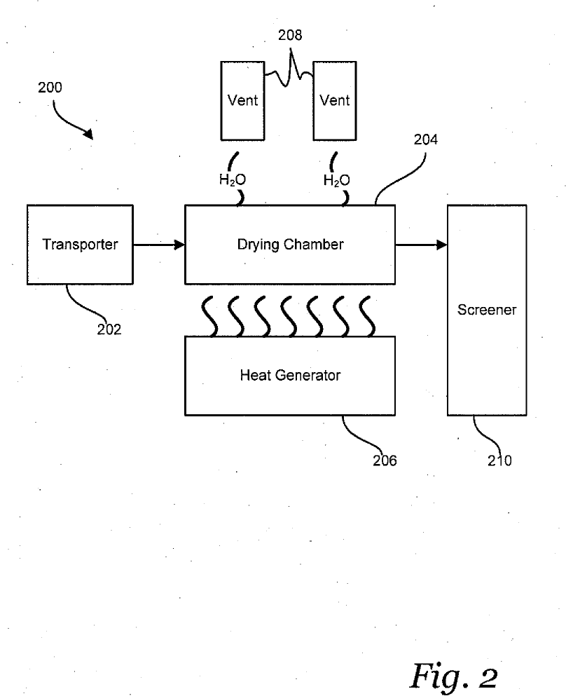

According to embodiments, a dryer is illustrated in FIG. 2. According to the embodiment, raw PCC is transported to dryer 200 with transporter 202. Transporter 202 may comprise a lead screw, for example. Transporter 202 may also comprise a pump and conduit system or conveyer belt-type apparatus depending on the moisture level of the raw PCC. According to still other embodiments, transported may comprise a waste (PCC) removal system in the sugaring process of sugar beets, whereby the PCC is deposited directly from the sugaring process into drying chamber 204. Other embodiments include manually loading raw PCC into dryer. Raw PCC exits transporter 202 into drying chamber 204, where the PCC is heated.

Drying chamber 204 is comprised of a lead screw in a U-trough surrounded by heat generated by heat generator 206, according to embodiments. Use of a lead screw is effective because it mixes the PCC improve the efficiency of the drying process. According to embodiments, PCC that has passed through drying chamber 204 will have a moisture content of less than 2%. Artisans, however, will readily understand that any range of drying whereby the moisture content of the PCC is between 0% and 75% may be useful according to embodiments. Generally, when the PCC is used as a cement replacement, it should meet a minimum dryness level of between 0% and 10% to prevent premature chemical reaction of the cement or concrete. An effective moisture content range of the PCC after drying is between 0% and 5%. Another effective moisture range of the PCC after the drying process has been found to be between 0% and 2%.

According to embodiments, drying chamber 204 provides controlled heat and ventilation. Accordingly, heat source 206 comprises a steam generator, according to embodiments that surrounds drying chamber 204, but is not in fluid or gaseous communication with drying chamber 204. Thus, heat source 206 would work according to the principles of a heat exchanger. According to still other embodiments, heat source 206 comprises a furnace which provides hot, dry air to drying chamber 204. According to still other embodiments, warm or hot off gasses or fluid from the sugar process of sugar beets may be used, either in gaseous communication or not in fluid or gaseous communication, to heat drying chamber 204.

According to embodiments, the energy used to heat drying chamber 204 is derived from biogas, steam, coke, electricity, solar, or natural gas. Drying chamber 204 is heated externally or heat can be forced through drying chamber 204, as described and well understood by artisans, according to embodiments. Temperatures within drying chamber should range 80 to 400 degrees Fahrenheit, depending on final moisture content of the PCC desired, the time the PCC spends in drying chamber 204, and the volume of PCC in drying chamber 204. The exact temperature, taking in account all of the factors listed herein may be determined on a case-by-case basis without undue experimentation.

As the PCC is heated, the moisture content is vaporized and is vented through vent 208. Vent 208 comprises any known conventional vent that allows for water vapor to be released from drying chamber 204 such that additional moisture may be absorbed into the gas in drying chamber 204. According to other embodiments, drying chamber 204 may be open to the ambient atmosphere, which will have sufficient vapor load capacity to substantially absorb all vaporized moisture content from the drying PCC.

According to embodiments, drying 200 includes a two step process. The first step, according to embodiments, is air drying. During air drying, a large portion of the moisture in the raw PCC is removed into the air. The second step comprises heated drying as described above.

After drying of the raw PCC, it is fed into screener 210. Feeding process may comprise any of the previously disclosed methods for moving PCC including, but not limited to, lead screws, conveyor belt-type mechanisms, manually, etc.

Turning again to FIG. 1, after drying in operation 106, the raw PCC is screened in operation 108. Screening in operation 108 filters out larger particles in the raw PCC to form a finished PCC suitable for use in cement or concrete applications. Depending on the particular application in cementitious use, the PCC is screened, generally from 200 to 400 minus. Screening, however, may be accomplished down to the angstrom level, for example 100 angstroms. Smaller PCC particles enhance the particle packing optimization in cementitious material distribution, which is highly desirable for CaCO₃ replacement. After screening, the refined PCC is used as a cementitious replacement product in operation 110, as known and understood by artisans, but generally to replace mined lime as a raw material in the cement or concrete.

According to embodiment the screening process of operation 108 (FIG. 1) separates particles of different sizes into separate aliquots using multiple screening steps. For example, 400 minus is separated into one group and 200 minus particles are separated into a separate group. The dried and screened PCC will then be ready for storage or transport for use in the cementitious process.

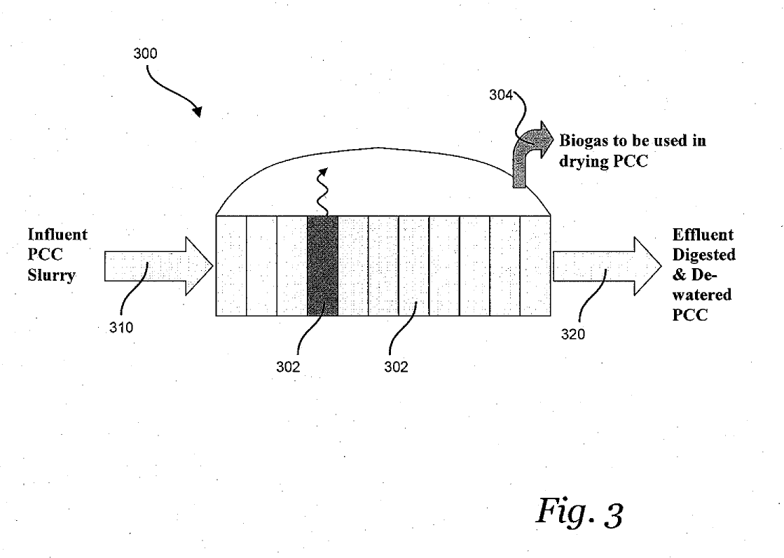

According to embodiments and as illustrated in FIG. 3, raw PCC is biodigested prior to being dried (operation 104 of FIG. 1). According to embodiments, the raw PCC solution is placed into a biodigestion chamber. Biodigestion allows for decomposition of organic materials in raw PCC collected during the sugaring process, improving the purity of the finished PCC. According to embodiments, biogases generated during the PCC process are captured and subsequently used as an energy source in dryer 200.

Alternatively, if the PCC is already air dried, it will be moved directly into forced heat dryer and treated the same as above.

According to embodiments and as illustrated in FIG. 3, biodigester 300 is shown for the biodigestion of organic molecules. Raw PCC is loaded into biodigestion chamber 302. Biodigestion chamber 302 comprises one of a plurality of biodigestion chambers that moves through biodigester 300 over a time period, according to embodiments. According to alternative embodiments, biodigestion chamber 302 comprises both passive and active transport methods including but not limited to a single conveyer belt-like mechanism or lead screw, whereby the raw PCC is placed on the conveyor belt-like mechanism or lead screw and is moved through biodigester over a time period.

Accordingly, raw PCC enters biodigester 300 at a location represented by arrow 310 and flows out of biodigester 300 at a location represented by arrow 320. Artisans will appreciate that the location of either arrow 310, 320 is arbitrary provide raw PCC is in biodigester 300 for a long enough time period whereby biodigestion occurs. During the time the PCC is in biodigester 300, anaerobic microorganisms digest organic molecules in the raw PCC. Biogases 304 is emitted as a product of the biodigestion process, which may be used in the drying process as described previously.

The advantages of the present disclosure include, without limitation, gives a large use stream for the waste PCC, decreases the need for environmentally unsustainable cement, savings through cement content reduction, improves green strength, provides better fluidity, workability, and finish of the cementitious mix, minimizes excessive bleeding and segregation due to improved particle packing, better fire resistance compared to fly ash concrete, reduces pumping energy and abrasion, and decreases efflorescence in concrete inter alia.

It is well within the skill of a person in the technical field, upon becoming conversant with, or otherwise having knowledge of, the present disclosure, to select suitable combinations of components such as the drying, screening, and mixing of PCC being designed or constructed.

While the apparatus and method have been described in terms of what are presently considered to be the most practical and preferred embodiments, it is to be understood that the disclosure need not be limited to the disclosed embodiments. It is intended to cover various modifications and similar arrangements included within the spirit and scope of the claims, the scope of which should be accorded the broadest interpretation so as to encompass all such modifications and similar structures. The present disclosure includes any and all embodiments of the following claims.

1. A method comprising:
obtaining raw precipitated calcium carbonate (PCC);
drying the PCC;
screening the PCC to form a finished PCC;
providing the finished PCC to be used as a cementitious replacement.

2. The method of claim 1, wherein the raw PCC is obtained from the sugaring process of sugar beets.

3. The method of claim 1, wherein the raw PCC is dried to a moisture level of less than 75%.

4. The method of claim 3, wherein the moisture level is lesser than or equal to 10%.

5. The method of claim 4, wherein the moisture level is lesser than or equal to 2%.

6. The method of claim 1, wherein the PCC is screened to from 200 minus to 100 angstroms.

7. The method of claim 1, wherein the finished PCC is provided to replace cementitious materials in concrete or cement.

8. The method of claim 1, further comprising biodigesting the raw PCC.

9. The method of claim 8, wherein the biogas is used as an energy source to dry the PCC.

10. An apparatus comprising:
a collector for collecting raw precipitate calcium carbonate (PCC) from a process producing as a waste product;
a dryer for removing the moisture content of the PCC to a predetermined level; and
a screener for obtaining a finished PCC having a predetermined particle size suitable as a cementitious replacement;
wherein the raw PCC is processed to be used as a cementitious replacement.

11. The apparatus of claim 10, further comprising a biodigester.

12. The apparatus of claim 11, wherein the biogas generated by biodigestion in the biodigester is used as an energy source for the dryer.

13. The apparatus of claim 10, wherein the finished PCC is used to replace lime in concrete or cement.

14. The apparatus of claim 10, wherein the process comprises at least the sugaring of sugar beets.

15. The apparatus of claim 10, wherein the moisture level is no greater than 10%.

16. The apparatus of claim 10, wherein the PCC is screened to from 200 minus to 100 angstroms.

17. A method comprising:
obtaining raw precipitated calcium carbonate (PCC) from the sugaring process of sugar beets;
drying the PCC to a moisture level of not greater than 10%;
screening the PCC to form a finished PCC;
providing the finished PCC as a cementitious replacement.

18. The method of claim 17, wherein the PCC is screened to from 200 to 400 minus.

19. The method of claim 1, further comprising biodigesting the raw PCC.

20. The method of claim 1, wherein the finished PCC is provided to replace lime in concrete or cement.
